(19) United States
(12) Patent Application Publication
(10) Pub. No.: US 2010/0125050 A1
(43) Pub. Date: May 20, 2010

(54) Process for the Manufacture of Eptifibatide

(75) Inventor: Andre' Collin, Brussels (BE)

Correspondence Address: Solvay;c/o B. Ortego - IAM-NAFTA, 3333 Richmond Avenue, Houston, TX, 77098-3099, US

(73) Assignee: SOLVAY, SA, Brussels (BE)

(21) Appl. No.: 11/995,476

(22) PCT Filed: Jul. 14, 2006
(86) PCT No.: PCT/EP06/64253

§ 371 Date: Oct. 30, 2008

Related U.S. Application Data
(60) Provisional application No. 60/699,818, filed on Jul. 15, 2005.

(51) Int. Cl. A61K 38/12; A61P 9/00

(52) U.S. Cl. 514/11

(57) ABSTRACT

Process for manufacturing eptifibatide which comprises subjecting a solution of eptifibatide derivative in a solvent to a concentration step, wherein the pH of said solution is less than or equal to 7.

The present invention relates to a process for the manufacture of eptifibatide, in particular to such a process comprising a purification step.

Eptifibatide, which is commercialised by MILLENNIUM PHARMACEUTICALS under the name Integrelin™ is a cyclical heptapeptide of sequence

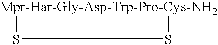

that selectively blocks the platelet glycoprotein IIb/IIIa receptor. It reversibly binds to platelets and has a short half-life. It has demonstrated efficacy as an intravenous solution in the treatment of patients during coronary angioplasty, myocardial infarction and angina.

Callens discloses in the IBC talk, San Diego, 1999 a method of manufacturing eptifibatide.

Scarborough et al. U.S. Pat. No. 5,686,571 discloses laboratory-scale chromatography of eptifibatide by reversed phase HPLC using a gradient of acetonitrile in trifluoroacetic acid, wherein the chromatography fractions are directly lyophilized.

It was desirable to have available a process which allows for manufacture of eptifibatide having required purity for its applications and which presents advantages in terms of productivity and required manufacturing equipment.

In consequence, the invention concerns a process for manufacturing eptifibatide which comprises subjecting a solution of eptifibatide derivative in a solvent to a concentration step, wherein the pH of said solution is less than or equal to 7.

It has been found, surprisingly, that the process according to the invention allows to provide concentrated pure eptifibatide material without substantial formation of by-products, in particular dimerization of eptifibatide, during the concentration step. It also allows in particular to meet specifications concerning purification related organic impurities such as for example organic solvents, in particular acetonitrile.

In the present invention, “solution of eptifibatide derivative” is intended to denote a substantially homogeneous, preferably a totally homogeneous liquid phase containing eptifibatide derivative dissolved therein.

In the present invention, “eptifibatide derivative” is intended to denote in particular a salt form of the cyclic heptapeptide such as chloride, trifluoroacetate or acetate. The preferred eptifibatide derivative is the acetate which is the commercialized drug.

In the process according to the invention the pH of the solution is preferably less than or equal to 5 and more preferably less than or equal to about 4.6. In the process according to the invention, the pH of the solution is generally more than or equal to 3 and preferably more than or equal to 4.

In the process according to the invention the pH of the solution is preferably maintained at the values indicated above throughout the concentration step. It is not preferred to work outside the indicated values though it might be possible to do so for a short period of time, without materially affecting the results obtained by virtue of the teaching of the present invention.

In the process according to the invention the pH of the solution is preferably maintained within a given interval around a chosen target pH value, as described above. Preferably, this interval is about ±0.5. The pH is generally maintained by measuring and adjusting, if necessary, by addition of pH controlling agent. A preferred pH controlling agent is an acetate buffer, for example in particular a mixture of eptifibatide acetate and suitably controlled amount of acetic acid. The pH controlling agent can be added to the solution before or during the concentration step.

In the process according to the invention, the concentration step is generally carried out at a temperature less than or equal to 40° C. preferably less than or equal to 35° C. and more preferably less than or equal to 30° C. In the process according to the invention, the concentration step is generally carried out at a temperature higher than or equal to −20° C. preferably higher than or equal to 0° C. and more preferably higher than or equal to 20° C. A concentration step carried out at a temperature of about 30° C. is more particularly preferred.

In the process according to the invention, the temperature is preferably maintained at the values indicated above throughout the concentration step. It is not preferred to work outside the indicated values though it might be possible to do so for a short period of time, without materially affecting the results obtained by virtue of the teaching of the present invention.

In the process according to the invention the temperature during the concentration step is preferably maintained within a given interval around a chosen target temperature value, as described above. Preferably, this interval is about ±5° C. and more preferably ±2° C.

In a particular embodiment of the process according to the invention the solvent comprises water and a polar organic co-solvent. Examples of polar organic co-solvent include organic nitriles and alcohols. Organic nitriles are preferred, acetonitrile is more particularly preferred. Among alcohols, alkyl alcohols, for example C1-C4 alcohols are preferred. Methanol and ethanol, in particular methanol can be suitably used.

In this embodiment the volume ratio of water to polar organic co-solvent is generally equal to or higher than 50:50 relative to the mixture water/polar organic co-solvent. Preferably this ratio is equal to or higher than 70:30. In this embodiment, the volume ratio of water to polar organic co-solvent is generally equal to or lower than 90:10 relative to the mixture water/polar organic co-solvent. Preferably this ratio is equal to or lower than about 80:20.

In this process according to the invention, the solvent preferably comprises acetic acid. When the solvent contains water and a polar organic co-solvent, the acetic acid content is preferably 0.1-1 volume parts per 100 volume parts of water/polar organic co-solvent mixture and more preferably about 0.3 volume parts per 100 volume parts of water/polar organic co-solvent mixture.

In the process according to the invention, the eptifibatide derivative contained in the solution preferably consists essentially of eptifibatide (acetate).

In the process according to the invention, the purity of the eptifibatide derivative in the solution subjected to the concentration operation is generally at equal to or higher than 98.5 area % determined by HPLC. Preferably this purity is equal to or higher than 99 area % and more preferably equal to or higher than 99.5 area %. Generally, the purity of the eptifibatide derivative in the solution subjected to the concentration operation does not exceed 99.7 area % determined by HPLC.

In the process according to the invention, the eptifibatide derivative concentration in the solution subjected to the concentration operation is generally equal to or higher than 0.5 wt. %, preferably equal to or higher than 1 wt. % and more preferably equal to or higher than 2 wt. %. In the process according to the invention, the eptifibatide derivative concentration in the solution subjected to the concentration operation is generally lower than 4 wt. %, preferably equal to or lower than 3 wt. %.

In the process according to the invention, the concentration step is frequently selected from an evaporation step, a reversed osmosis step and a precipitation or crystallization step. Preferably, the concentration step is selected from an evaporation step and a reversed osmosis step, and more preferably it is an evaporation step.

In particular when the concentration step is selected from an evaporation step and a reversed osmosis step, the material recovered from the concentration operation is generally a concentrated solution of eptifibatide derivative.

In this embodiment, the concentrated eptifibatide derivative solution recovered from the concentration operation has preferably an eptifibatide derivative concentration below the solubility limit of the eptifibatide derivative in the solvent having been subjected to evaporation or reversed osmosis, at the temperature of the concentration step. Preferably, the solution has a concentration of at least 70% molar of the solubility limit of the eptifibatide derivative in the solvent having been subjected to evaporation or reversed osmosis, at the temperature of the concentration step, more preferably at least 75% molar and most preferably about 80% molar of the solubility limit. It generally does not exceed about 85% molar of the solubility limit. It has been found that limiting the concentration step to this value allows for improved control of the process, thus avoiding undesired precipitation and associated loss of product lots. In a less advantageous embodiment, the eptifibatide derivative concentration is substantially below that solubility limit.

When a mixture of water and acetonitrile is used, the concentrated eptifibatide derivative solution has generally an eptifibatide derivative concentration equal to or lower than the about 5 wt. % but preferably equal to or higher than 4 wt. %.

In the process according to the invention, the purity of the eptifibatide derivative in the concentrated material recovered from the concentration operation is generally at equal to or higher than 98.5 area % determined by HPLC. Preferably this purity is equal to or higher than 99 area % and more preferably equal to or higher than 99.5 area %. Generally, the purity of the eptifibatide derivative in the concentrated material recovered from the concentration operation does not exceed 99.7 area % determined by HPLC.

In the process according to the invention, the content of dimeric eptifibatide derivatives in the concentrated material recovered from the concentration operation is generally equal to or lower than 0.2 area % determined by HPLC, preferably equal to or lower than 0.1 area %. If dimeric eptifibatide derivatives are present in the concentrated material recovered from the concentration operation, their concentration can be at least 0.05 area % determined by HPLC.

In a preferred embodiment of the process according to the invention, the concentration step comprises evaporating solvent from the solution. In this embodiment, the solvent is evaporated generally evaporated at a pressure higher than or equal to 0.001 bars, preferably higher than or equal to 0.01 bars. In this embodiment, the solvent is generally evaporated at a pressure lower than or equal to 1 bar, preferably lower than or equal to 0.5 bars, more preferably lower than or equal to 0.1 bars.

When a mixture of water and acetonitrile is used in this preferred embodiment, the evaporation comprises evaporating a mixture of water and acetonitrile, whereby the concentration of acetonitrile in the solution is reduced.

In a particularly preferred aspect of this embodiment, water is added to the solution during the evaporation step so as to further reduce the content and preferably substantially eliminate acetonitrile in the concentrated solution. Typical acetonitrile contents in the concentrated solution are less than about 100 mg/kg, preferably less or equal than about 30 mg/kg and most preferably less or equal than about 20 mg/kg.

In a preferred embodiment, the process according to the invention comprises steps (a) to (d) wherein
(a) crude eptifibatide derivative is manufactured by coupling of peptide fragments, for example according to the reaction sequence disclosed in Callens, IBC Talk cited above;
(b) the crude eptifibatide derivative is subjected to a purification operation comprising at least one liquid chromatography operation to obtain a solution of eptifibatide derivative;
(c) the solution of eptifibatide derivative is subjected to the concentration step according the invention to obtain material having increased concentration of eptifibatide; and
(d) the material having increased concentration of eptifibatide is subjected to a lyophilization operation to obtain eptifibatide.

The example here after is intended to illustrate the invention without however limiting its scope.

### EXAMPLE

80 l of a solution of eptifibatide acetate having a purity of 99.5% measured by analytical HPLC on a C-18 reversed phase column, having an eptifibatide concentration of 15.2 g/l in a mixture water/acetonitrile/acetic acid 70 parts by volume/30 parts by volume/0.3 parts by volume said solution having a pH of 4.6. was introduced into a tubular climbing film evaporator and an acetonitrile/water mixture was evaporated at a temperature kept at about 30° C. During the evaporation, 30 l of water were fed continuously at the rate of evaporation. The evaporation was then continued until the volume of the solution had decreased to about 30 l. The pH of this solution was 4.2. A concentrated solution of eptifibatide acetate having a concentration of 40.5 g/l a and purity of 99.5% and no detectable dimer was recovered and introduced into a lyophilization step. Eptifibatide acetate having appropriate purity for use in medicament was obtained in high yield from that step.

The process according to the invention allows to reduce the volume of solution introduced into further isolation procedures such as cost-intensive lyophilization step while maintaining required purity of eptifibatide derivative for pharmaceutical application and substantially avoiding formation of by-products, in particular dimeric by-products.

1. A process for manufacturing eptifibatide which comprises subjecting a solution of eptifibatide derivative in a solvent to a concentration step, wherein the pH of said solution is less than or equal to 7.

2. The process according to claim 1, wherein the pH of the solution is less than or equal to 5.

3. The process according to claim 1 or 2, wherein the pH of the solution is more than or equal to 3.

4. The process according to anyone of claims 1 to 3, wherein concentration step is carried out at a temperature less than or equal to 40° C.

5. The process according to claim 4, wherein the temperature is from 0 to 35° C.

6. The process according to claim 5, wherein the temperature is from 20 to 35° C.

7. The process according to anyone of claims 1 to 6, wherein the concentration step comprises evaporating solvent from the solution.

8. The process according to claim 7, wherein the solvent is evaporated at a pressure from 0.001 to 0.5 bars.

9. The process according to anyone of claims 1 to 8, wherein the solvent comprises water and a polar organic co-solvent.

10. The process according to claim 9 which comprises evaporating a mixture of water and polar organic co-solvent.

11. The process according to anyone of claims 1 to 10, wherein the solvent comprises acetic acid.

12. The process according to anyone of claims 1 to 11, wherein the eptifibatide derivative contained in the solution consists essentially of eptifibatide (acetate).

13. The process according to anyone of claims 1 to 12, wherein the eptifibatide derivative concentration in the solution subjected to the concentration operation is from 0.5 to 3 wt. %.

14. The process according to anyone of claims 1 to 13, wherein the material recovered from the concentration operation is a concentrated solution of eptifibatide derivative.

15. The process according to claim 14, wherein the concentrated solution has an eptifibatide derivative concentration of from 4 to 5 wt. %.

16. The process according to anyone of claims 1 to 15, which comprises steps (a) to (d) wherein
(a) crude eptifibatide derivative is manufactured by coupling of peptide fragments;
(b) the crude eptifibatide derivative is subjected to a purification operation comprising at least one liquid chromatography operation to obtain a solution of eptifibatide derivative;
(c) the solution of eptifibatide derivative is subjected to the concentration step according to anyone of claims 1 to 15 to obtain material having increased concentration of eptifibatide; and
(d) the material having increased concentration of eptifibatide is subjected to a lyophilization operation to obtain eptifibatide.

17. A solution of an eptifibatide derivative which contains from 0.5% wt. to 3% wt. of eptifibatide derivative in a solvent comprising water, a polar organic co-solvent and acetic acid.

18. The solution according to claim 17, which contains from 0.1 to 1 volume part of acetic acid per 100 volume parts of mixture of water/polar organic co-solvent.

19. The solution according to claim 17 or 18, wherein the polar organic co-solvent is acetonitrile.

20. The solution according to anyone of claims 17 to 19, wherein the eptifibatide derivative is eptifibatide (acetate).
